(19) United States
(12) Patent Application Publication
(10) Pub. No.: US 2008/0306584 A1
(43) Pub. Date: Dec. 11, 2008

(54) Implantable medical devices for local and regional treatment

(76) Inventor: Pamela Kramer-Brown, San Jose, CA (US)

Correspondence Address: SQUIRE, SANDERS & DEMPSEY LLP, 1 MARITIME PLAZA, SUITE 300, SAN FRANCISCO, CA, 94111, US

(21) Appl. No.: 11/810,518

(22) Filed: Jun. 5, 2007

(51) Int. Cl. A61F 2/06

(52) U.S. Cl. 623/1.39; 977/931

(57) ABSTRACT

Implantable medical devices adapted to erodibly release delivery media for local and regional treatment are disclosed.

### BACKGROUND OF THE INVENTION

1. Field of the Invention

This invention relates to implantable medical devices adapted to deliver media for local and regional treatment.

2. Description of the State of the Art

This invention relates generally to implantable medical devices for treating bodily disorders. A typical treatment regimen with an implantable medical device involves implantation of a device at a selected treatment location. During treatment it may be necessary for the device to support body tissue. Therefore, the structure of a device may include load bearing structural elements or substrate to hold the device in place and to resist forces imposed by surrounding tissue.

The treatment of a bodily disorder may also involve local delivery of a bioactive agent or drug to treat a bodily disorder. The agent may be incorporated into the device in a variety of ways and delivered directly to an afflicted region at or adjacent to a region of implantation.

Additionally, in many treatment situations, the presence of the device is required only for a limited period of time. Therefore, a device may be composed in whole or in part of materials that degrade, erode, or disintegrate through exposure to conditions within the body until the treatment regimen is completed.

An example of such devices includes radially expandable endoprostheses, which are adapted to be implanted in a bodily lumen. An “endoprosthesis” corresponds to an artificial device that is placed inside the body. A “lumen” refers to a cavity of a tubular organ such as a blood vessel.

A stent is an example of such an endoprosthesis. Stents are generally cylindrically shaped devices, which function to hold open and sometimes expand a segment of a blood vessel or other anatomical lumen such as urinary tracts and bile ducts. Stents are often used in the treatment of atherosclerotic stenosis in blood vessels. “Stenosis” refers to a narrowing or constriction of the diameter of a bodily passage or orifice. In such treatments, stents reinforce body vessels and prevent restenosis following angioplasty in the vascular system. “Restenosis” refers to the reoccurrence of stenosis in a blood vessel or heart valve after it has been treated (as by balloon angioplasty, stenting, or valvuloplasty) with apparent success.

The treatment of a diseased site or lesion with a stent involves both delivery and deployment of the stent. “Delivery” refers to introducing and transporting the stent through a bodily lumen to a region, such as a lesion, in a vessel that requires treatment. “Deployment” corresponds to the expanding of the stent within the lumen at the treatment region. Delivery and deployment of a stent are accomplished by positioning the stent about one end of a catheter, inserting the end of the catheter through the skin into a bodily lumen, advancing the catheter in the bodily lumen to a desired treatment location, expanding the stent at the treatment location, and removing the catheter from the lumen.

In the case of a balloon expandable stent, the stent is mounted about a balloon disposed on the catheter. Mounting the stent typically involves compressing or crimping the stent onto the balloon. The stent is then expanded by inflating the balloon. The balloon may then be deflated and the catheter withdrawn. In the case of a self-expanding stent, the stent may be secured to the catheter via a retractable sheath or a sock. When the stent is in a desired bodily location, the sheath may be withdrawn which allows the stent to self-expand.

The stent must be capable of withstanding the structural loads, namely radial compressive forces, imposed on the stent as it supports the walls of a vessel. Therefore, a stent must possess adequate radial strength, which is the ability of a stent to resist radial compressive forces. Once expanded, the stent must adequately maintain its size and shape throughout its service life despite the various forces that may come to bear on it, including the cyclic loading induced by the beating heart. In addition, the stent must possess sufficient flexibility to allow for crimping, expansion, and cyclic loading.

The structure of a stent is typically composed of scaffolding or substrate that includes a pattern or network of interconnecting structural elements often referred to in the art as struts or bar arms. The scaffolding can be formed from wires, tubes, or sheets of material rolled into a cylindrical shape. The scaffolding is designed so that the stent can be radially compressed (to allow crimping) and radially expanded (to allow deployment).

Additionally, a drug-eluting stent may be fabricated by coating the surface of either a metallic or polymeric scaffolding with a polymeric carrier that includes an active or bioactive agent or drug. Polymeric scaffolding may also serve as a carrier of an active agent or drug. Currently drugs or drug mixtures are typically released from coatings through diffusion or elution through coating. In addition, for pure drugs dispersed in coatings, the time frame of the therapeutic effect of the drug is relatively short. As a result, the treatment is limited to a region local to the region of implantation of the stent.

In many treatment applications, the presence of a stent in a body may be necessary for a limited period of time until its intended function of, for example, maintaining vascular patency and/or drug delivery is accomplished. Therefore, stents fabricated from biodegradable, bioabsorbable, and/or bioerodable materials such as bioabsorbable polymers can be configured to completely erode after the clinical need for them has ended.

In some treatment situations, local treatment of bodily tissue disorders with an implantable medical device may be difficult or insufficient. This insufficiency may be from the fact that tissue disorders may be diffuse and in multiple locations. Local treatment in such situations may require a multiplicity of devices. For example, vascular disorders can include lesions in multiple locations, such as diffuse lesions along vessels, multi-vessel lesions, and bifurcated vessel lesions. In addition, local treatment may be impossible because an afflicted region of tissue may be inaccessible to implantation of a device. For example, a diseased vessel may be too small for implantation of a stent. Thus, it would be desirable to have an implantable medical device that can be used to treat tissue disorders both local and regional to the location of implantation.

### SUMMARY OF THE INVENTION

Certain embodiments of the present invention include a stent comprising a scaffolding formed from a corrodible metal having one or more recesses in a surface of the scaffolding, the recesses being at least partially filled with a plurality of releasable delivery media comprising an active agent, wherein the active agent is adapted to be released from the delivery media upon release of the delivery media from an implanted stent.

### BRIEF DESCRIPTION OF THE DRAWINGS

FIG. 1 depicts a view of a stent.

FIG. 2A depicts a section of a blood vessel with an implanted stent.

FIG. 2B depicts an expanded portion of an interface between an erodible matrix of a stent having embedded delivery particles.

FIG. 3 depicts a cross-section of a strut of a stent illustrating the geometry of an exemplary depot.

FIGS. 4A-B illustrate a cross-sections of struts with a depot filled with a delivery media.

FIGS. 5A-B is a schematic illustration of an expanded section of a delivery media showing particles of delivery media.

FIG. 6A depicts an overhead view of a stent strut with a well containing active agent or delivery media.

FIG. 6B depicts a side view of the strut of FIG. 6A showing a coating layer disposed above the well.

FIG. 7A depicts a delivery media layer over a corrodible metallic substrate.

FIG. 7B depicts an expanded portion of the layer in FIG. 7A.

FIG. 7C a topcoat layer over a delivery media layer over a corrodible metallic substrate.

FIG. 8 depicts a cross-section of a strut of a stent with three polymer layers.

FIG. 9 depicts a cross-section of a layered strut.

FIG. 10 depicts a cross-section of a three layer strut.

FIG. 11A depicts a cross-section of a three layer strut with a center layer partially eroded.

FIG. 11B depicts a cross-section of a layered strut after collapse of a middle layer.

### DETAILED DESCRIPTION OF THE INVENTION

Embodiments of the present invention can generally be applied to implantable medical devices including, but is not limited to, self-expandable stents, balloon-expandable stents, stent-grafts, vascular grafts other expandable tubular devices for various bodily lumen or orifices. The embodiments can be used in the local and regional treatment bodily disorders in various bodily lumens, including, but not limited to vulnerable plaque, atherosclerotic progression, and diabetic nephropathy.

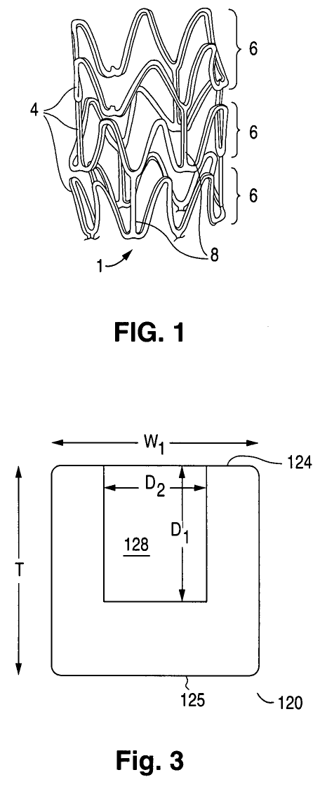

FIG. 1 depicts a view of a stent 1 which is made up of struts 4. Stent 1 has interconnected cylindrical rings 6 connected by linking struts or links 8. The embodiments disclosed herein are not limited to stents or to the stent pattern illustrated in FIG. 1. The embodiments are easily applicable to other stent patterns and other devices. The variations in the structure of patterns are virtually unlimited.

A stent such as stent 1 may be fabricated from a tube by forming a pattern with a technique such as laser cutting. Representative examples of lasers that may be used include an excimer, carbon dioxide, and YAG. In other embodiments, chemical etching may be used to form a pattern on the elongated tube.

As discussed above, the current state of the art includes a drug-eluting stent that has a coating on its surface with a polymeric carrier that includes an active or bioactive agent or drug dispersed in pure form throughout the carrier. Upon implantation, the active agent diffuses or elutes through the carrier and is released into a lumen. The therapeutic effect of the eluted agent is limited to the region immediately adjacent to the implanted stent.

Various embodiments of the present invention relate to implantable medical devices, such as a stent, for treating bodily tissue disorders with therapeutic agents both locally and regionally. Regional treatment refers to treatment of regions of bodily tissue that are proximal and/or distal to an implantation site. In some embodiments, the stent can be biodegradable so that it can disintegrate and disappear from the region of implantation once treatment is completed.

In some embodiments, a plurality of releasable delivery media may be incorporated within or on an implantable medical device. The delivery media can be released from the stent upon implantation. In certain embodiments, the delivery media can be transported distal to the implant site. An active agent incorporated in or on the delivery media may released from the delivery media in a sustained manner. As a result, delivery from the delivery media can occur both locally and regionally over an extended time frame.

As discussed in more detail below, a delivery medium can be, for example, a particle with an active agent encapsulated or dispersed within, adsorbed to the surface of or absorbed within the outside surface of the delivery particle. Alternatively, the delivery particle may be formed by a precipitate of a bioactive agent, e.g., by a neat bioactive agent or a salt of the bioactive agent with low solubility. The active agent included can be released from the delivery media into a patient's body after release of the delivery media from the device. The delivery media allows for sustained-release of active agent from the delivery media into the body after release of the delivery media from the stent implant.

As used herein, the term “sustained release” generally refers to a release profile of an agent or drug that can include zero-order release, exponential decay, step-function release or other release profiles that carry over a period of time, for example, ranging from several hours to several years, preferably from several days to several months, most preferably from several days to several weeks. The terms “zero-order release”, “exponential decay” and “step-function release” as well as other sustained release profiles are well known in the art (see, for example, Encyclopedia of Controlled Drug Delivery, Edith Mathiowitz, Ed., Culinary and Hospitality Industry Publications Services).

Delivery media may be incorporated into or onto a stent implant in various ways, as described in more detail herein. For example, the media can be disposed within depots or holes at the surface of the substrate, disposed in a coating on the surface of the substrate, or embedded or dispersed in the substrate of the stent implant. In one embodiment, the release of the media may be due in whole or in part to erosion or degradation of coating material, substrate material, or material which binds the delivery media to or within the stent implant. In further embodiments, the released media can be transported away from a region of implantation to a distal and/or proximal region after being released. The active agent can be released from the media during transport resulting in treatment of distal and/or proximal regions with the active agent.

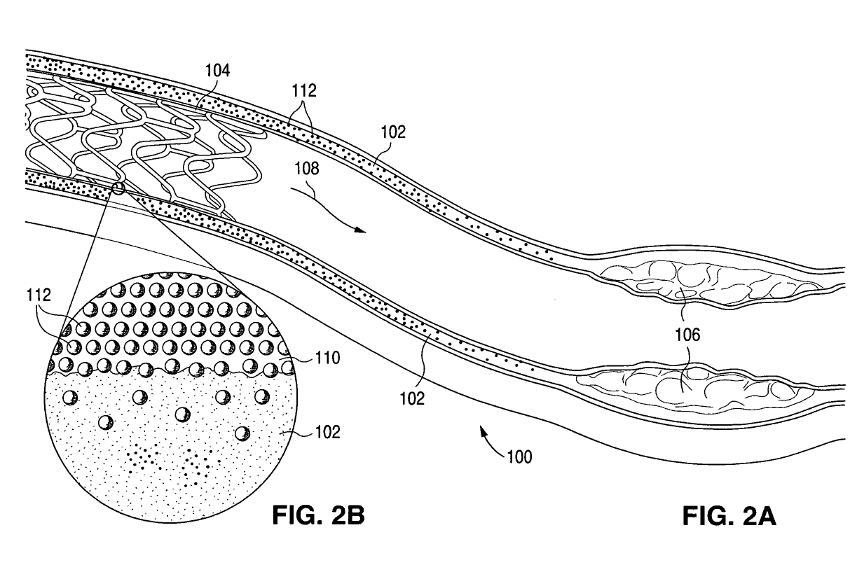

FIGS. 2A and 2B provide a schematic illustration of regional treatment with a stent. FIG. 2A depicts a section of a blood vessel 100 having vascular walls 102. A stent 104 is implanted distal to a non-flow limiting lesion 106. Delivery media, such as particles, can be selectively or directionally disposed on abluminal faces, luminal faces, both abluminal and luminal faces, and sidewalls of a stent. Selectively disposing particles in this manner allows for directional release of the particles and drug release to a targeted region. As depicted in FIG. 2A, the delivery particles 112 are released from stent 104 into the tissue of vascular wall 102. Particles can be selected that can diffuse through the tissue of vascular wall 102 and deliver both locally and to a distal and/or proximal region of vasculature, such as lesion 106.

FIG. 2B depicts an expanded portion of an interface between an erodible matrix 110 of stent 104 having embedded delivery particles. Erodible matrix 110 can be material disposed within a depot in stent 104, a coating over stent 104, or the scaffolding of stent 104.

Delivery particles can also be released into the blood stream for treatment of distal and/or proximal vasculature after implantation. Delivery particles can be released from the stent into the interior of the lumen, for example, from a luminal face of the stent. The released particles can be transported downstream as shown by an arrow 108 of the implanted stent 104 to a proximal or distal regions of vasculature, such as lesion 106. In some embodiments, particles may be designed to have or selected to have an affinity to a portion of a proximal or distal region of the vasculature. Such particles may selectively bind to a portion, e.g., by incorporating a peptide or an antibody fragment with affinity to receptors found on endothelial cells of the microvasculature into the surface of the particles.

In certain embodiments, the scaffolding or substrate of the implantable medical device can be fabricated from a biostable or non-corrodible material. Such a material can be a biostable polymer, non-corrodible metal, or a combination thereof.

As discussed above, an implantable medical device, such as a stent scaffolding or substrate, can be fabricated from a material that erodes or disintegrates upon implantation into the body. The terms degrade, absorb, and erode, as well as degraded, eroded, and absorbed, are used interchangeably and refer to materials that are capable of being completely eroded, or absorbed when exposed to bodily conditions. The term “corrosion” or “corrode” is typically used to refer erosion of a metal. Such materials may be capable of being gradually resorbed, absorbed, and/or eliminated by the body. A device made of such materials may disintegrate and disappear from a region of implantation once a treatment is completed.

The duration of a treatment period depends on the bodily disorder that is being treated. In treatments of coronary heart disease involving use of stents in diseased vessels, the duration can be in a range from about a week to a few years. However, the duration is typically in a range from about six to twelve months.

In certain embodiments, a stent scaffolding or substrate can be formed in whole or in part of a corrodible metal. The metal selected for use in an implantable medical device in accordance with the present invention may include a single element, such as iron, or may include a combination of metals. Generally, the metal(s) must be implantable without causing significant inflammation, neointimal proliferation or thrombotic events and must be corrodible so as to dissolve, dissociate or otherwise break down in the body without significant ill effect.

In one embodiment, the corrodible metal can be a metal that has a propensity for self-dissolution in an in vivo environment. A metal that undergoes self-dissolution in an in vivo environment corrodes when subjected to bodily fluids and breaks down. A self-dissolving metal can be selected that has little or no ill effect to a patient. Representative examples of self-dissolving metals in an in vivo environment include, but are not limited to, Mg, Mn, K, Ca, Na, Zn, Cr, Fe, Cd, Al, Co, Sb, Sn, V, Cu, W, and Mo.

Alternatively, the corridible metal may include a combination of two or more metals selected to create a galvanic couple such that the material will undergo galvanic dissolution upon contact with bodily fluids. Reliance on galvanic corrosion in order to achieve a desired corrosion rate requires the selection of a metal pair that has a sufficiently high rest potential differential. A rest potential differential results from two metals that, by themselves, each have a particular rest potential when measured versus a reference electrode, for example a Standard Calomel Electrode (SCE) or Natural Hydrogen Electrode (NHE), in the same type of solution, for example saline or equine horse serum. The driving force toward corrosion that results from this differential may be tailored to control the rate of degradation of the joined materials. For example, a driving force of about 500 mV would generally result in a slower dissolution than a driving force of 1 V or more. Appropriate metal pairs can be selected from among the elements Mg, Mn, K, Ca, Na, Zn, Cr, Fe, Cd, Al, Co, Sb, V, Cu, and Mo, and from alloys based on such elements.

The degradation rate may be tailored by selecting a combination of metals that have a driving force of about 500 mV or greater. In one embodiment the driving force would be about 1 V or greater. For example, Ti has a rest potential of 3.5 V vs. SCE in equine serum, and would, when paired with almost any other metal, yield a suitable driving force. Alternatively, the pairings Nb—Cr (1.1 V rest potential differential vs. SCE in equine serum), Pd—W (1.23 V rest potential vs. SCE in equine serum), Cr—W (630 mV rest potential differential vs. SCE in equine serum), and Ir—Zn (830 mV rest potential differential vs. SCE in equine serum) would also yield suitable driving forces.

In some embodiments, the stent can be formed of a porous corrodible metal. The pores increase the surface area of contact of bodily fluids which tends to accelerate the corrosion rate of the metal. By selecting the metal and the degree of porosity, the rates of degradation can be tailored to a range of applications. The porosity has a substantial effect on the rate of corrosion to the extent that the ratio of corrosion rate increase to surface area increase has been found to vary from 0.3 to 1.0 depending on the type of material and the environment to which it is exposed. The morphology of the microcellular porous metal, including the cell size and porosity of the metal, can be controlled so that the cell sizes can be made very uniform, and can be controlled precisely by the manipulation of various parameters during the formation process. The desired porosity is achievable by a variety of techniques including, but not limited to sintering, foaming, extrusion, thixomolding, semi-solid slurry casting and thermal spraying. The stent structure may be formed using any of the well known techniques, including, for example, laser cutting of a tubular form.

In some embodiment, a device, coating, or binder for the delivery media, or more specifically, particles, can be composed of a biodegradable or water soluble polymer. In general, polymers can be biostable, bioabsorbable, biodegradable, or bioerodable. Biostable refers to polymers that are not biodegradable. The terms biodegradable, bioabsorbable, bioerodable, and soluble, as well as degraded, eroded, absorbed, and dissolved are used interchangeably and refer to polymers that are capable of being completely eroded, absorbed, or dissolved after implantation, e.g., when exposed to bodily fluids such as blood and can be gradually resorbed, absorbed, and/or eliminated by the body. The mechanism of absorption or clearance is entirely different for a bioerodible versus a biosoluble polymer.

As discussed above, the delivery media can include particles that include active agent(s). The particles can be nanoparticles or microparticles. A nanoparticle refers to a particle with a characteristic length (e.g., diameter) in the range of about 1 nm to about 1,000 nm. A microparticle refers to a particle with a characteristic length in the range of greater than 1,000 nm and less than about 10 micrometers. Methods for the manufacture of microparticles are well known to those skilled in the art. Microparticles are commercially available from a number of sources (for example: Alkermes Inc. Cambridge Mass.).

Particles may have active agents mixed, dispersed, or dissolved in the particle material. The particle material can be a biostable or biodegradable polymer, metallic, or ceramic. Such particles may also be coated with an active agent. The particles can also encapsulate one or more active agents by having an outer shell of polymer, metal, or ceramic with an inner compartment containing one or more active agents. Alternatively, the particle may be formed from a precipitate of neat drug.

In some embodiments, particles may be designed to use a combination of the above, e.g., a particle may include a polymeric and a drug, or a drug- or agent-impregnated core coated with a bioerodible metal. In addition, particles may include fullerenes coated with a bioactive agent. Particles may also include polymerosomes, micelles, vesicles, liposomes, glass (biodegradable and biostable), and micronized drug.

Representative examples of materials that may be used for particles include, but are not limited to, a biostable polymer; a bioabsorbable polymer; a biosoluble material; a biopolymer; a biostable metal; a bioerodible metal; a block copolymer of a bioabsorbable polymer or a biopolymer; a ceramic material such as a bioabsorbable glass; salts; fullerenes; lipids; carbon nanotubes; or a combination thereof.

A “micelle” refers to an aggregate (or cluster) of surfactant molecules. “Surfactants” refer to chemicals that are amphipathic, which means that they contain both hydrophobic and hydrophilic groups. Micelles tend to form when the concentration of surfactant is greater than a critical micelle concentration. Micelles formed from block copolymers and/or lipids may be loaded with active agent. Micelles can exist in different shapes, including spherical, cylindrical, and discoidal. Micelles may be stabilized by crosslinking of the surfactant molecules that form the micelle.

Additionally, vesicles formed from block copolymers and or lipids can be loaded with bioactive agent. A vesicle is a relatively small and enclosed compartment or shell formed by at least one lipid bilayer. The vesicle may also be stabilized by crosslinking the lipid bilayer shell.

In some embodiments, delivery particles can be incorporated into a device substrate, coating, or depots in a substrate with a binder that holds the particles together within or on the device. In an embodiment, a surfactant may be utilized to enhance integration of the particles into the binder matrix. The binder may be composed in whole or in part of an erodible binder material. The particles may then be released from the device upon erosion of the binder material. Representative examples of materials that may be used for a binder include, but are not limited to, a bioabsorbable polymer; a biostable, but biosoluble polymer; a biosoluble material; a biopolymer; a biostable metal; a bioerodible metal; a block copolymer of a bioabsorbable polymer or a biopolymer; salts; bioerodible glass; or a combination thereof.

Additionally, delivery particles may be surface-modified to allow targeted delivery of biopharmaceuticals to bodily tissue. Such surface modification could be with antibodies or their fragments, small-molecular ligands, or specific receptors.

Various embodiments of the present invention include an implantable medical device, such as a stent implant, having releasable delivery media. Such delivery media provides sustained-release of active agent for treatment both locally and regionally to a site of device implantation.

Certain embodiments of a device can include a substrate or scaffolding of a stent formed from a corrodible metal having one or more recesses in a surface of the substrate. The recesses can be at least partially filled with delivery media that includes active agent(s). The delivery media allows for sustained release of an active agent from the media upon release of the media from the device.

Recesses can include, for example, depots or channels at a surface of a substrate of a device. Numerous embodiments of depots or channels configured to hold delivery media are possible. Depots, for example, may be placed at one or more arbitrary locations on a device. In addition to recesses, hollow struts could be configured to increase delivery media loading. Such hollow struts can be made by methods known by one of ordinary skill in the art.

FIG. 3 depicts a cross-section of a strut 120 of a stent illustrating the geometry of an exemplary depot 128 disposed at an abluminal face 124 of strut 120. Strut 120 has a width W. Depot 128 has a generally cylindrical shape with a depth D₁ and diameter D₂. The appropriate values for D₁ and D₂ depend on factors such as the effective delivery media, mechanical integrity of the strut, density of depots, and the desired time frame of release of the delivery media. For instance, the greater the effective amount of delivery media, and active agent(s) contained therein, the larger either or both depth D₁ and diameter D₂ may need to be. A higher density of depots disposed on a strut may decrease a required amount of delivery media in an individual strut, and thus a necessary size of a depot. Furthermore, as the size and density of the depots increase, the mechanical strength of the strut may decrease. Additionally, a longer sustained release of drug delivery media may be facilitated by a larger depth D₁. A diameter D₂ of cylindrical depot 128 may have a range from about 10% to about 95%, about 20% to about 80%, 30% to about 70%, or about 40% to about 60% of width W₁.

Additionally, the geometrical parameters that characterize depots such as size (e.g., depth, diameter, etc.) and shape may be configured to facilitate treatment of an inflammatory response. For example, the geometry of depots may be configured to maximize sustained delivery of anti-inflammatory agent throughout the degradation of a device to counteract the inflammatory effect of degradation by-products.

A single depot or plurality of depots may be formed as a laser trench or laser trenches on a body of an implantable medical device such as stent 1 by exposing a surface of the device to an energy discharge from a laser, such as an excimer laser. Alternative methods of forming depots include, but are not limited to physical or chemical etching techniques. Techniques of laser fabrication or etching to form depots are well-known to one of ordinary skill in the art. Depots can be formed in virtually any stent structure and not merely the above-described structure.

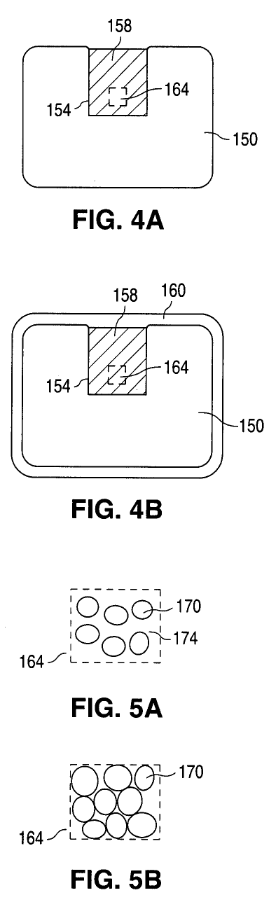

FIG. 4A illustrates a cross-section of a strut 150 with a depot 154 filled with delivery media 158. FIG. 4B illustrates another embodiment in which depot 158 can be covered by a coating 160. Coating 160 can be a degradable polymer coating that can delay the release of delivery media 158 from depot 154. Alternatively, a protective sleeve can be disposed over or within a stent to reduce or prevent premature delivery of the delivery media. The sleeve can be removed prior to or after implantation to allow erosion of the stent and delivery of the delivery media. The sleeve can be sized to have a slip or friction fit over a crimped stent. Such a sleeve could be made from biostable, biodegradable, or biosoluble polymers. In exemplary embodiments, the sleeve can be made of biostable elastomeric polymers such as poly ether block amides, for example, Pebax® from Arkema, Inc. of Philadelphia, Pa. In other exemplary embodiments, the sleeve can be formed from biodegradable elastomeric polymers such as polycaprolactone or poly(tetramethylene carbonate).

In some embodiments, coating 160 or a protective sleeve can include a dispersed active agent. The active agent(s) is the coating can be the same or different from the active agent in the delivery media. For example, in one embodiment, the delivery media can have an anti-inflammatory agent and the coating can have an anti-proliferative, or the reverse.

In certain embodiments, the delivery media can be incorporated into a depot with a binder that holds the individual particles of delivery media together and within the depot. FIG. 5A is a schematic illustration of an expanded section 164 of delivery media 158 showing particles 170 of delivery media that are dispersed within an erodible binder 174. The amount of delivery media can be varied through ratio of particles to binder material. For example, FIG. 5B depicts an embodiment showing particles 170 with little or no binder material. Such an embodiment may allow the largest amount of delivery media delivered to a patient. The binder material may be a coating on the surface of the particles that allows the particles to adhere to each other and the depot walls so that the particles remain in the depot at least until implantation of the stent. For example, the coating can include a hydrogel or a water soluble polymer. A coating over the opening of the depot can be used to contain particles having no binder material in the depot.

Since the particles are released as the binder material erodes or dissolves, the rate of the release of particles can be varied or controlled through selection of binder material. A fast eroding polymer or water soluble polymer can be selected to result in a fast or burst release of particles. A slower eroding polymer can be selected to obtain a slow or gradual release of particles. As mentioned above, the release of delivery media can be delayed by a coating layer over the opening of the depot, as depicted by coating 160 in FIG. 4B.

In alternative embodiments, the delivery media can be in the form of a suspension within a depot. For instance, delivery particles can be suspended within a fluid, such as an aqueous solution or other biocompatible fluid. In such an embodiment, the opening of the depot can be covered by an erodible coating, such as depicted by coating 160, to reduce or prevent flow of the suspension from the depot. The amount of delivery media can be varied through the ratio of particles to solution. The release profile in such embodiments can be configured to be a pulse release since the particles of delivery media may tend to rapidly flow out of the opening once a coating over the opening degrades away. “Pulse release” generally refers to a release profile that features a sudden surge of the release rate of the delivery media. The release rate surge of the delivery media would then disappear within a period. A more detailed definition of the term can be found in Encyclopedia of Controlled Drug Delivery, Edith Mathiowitz, Ed., Culinary and Hospitality Industry Publications Services.

In some embodiments, depots may be selectively distributed at or near portions of a surface of a stent depending upon the type of treatment desired. In such embodiments, a stent may have depots selectively distributed along a longitudinal axis. For example, a stent can have more depots or only have depots at a distal end, proximal end, or center portion.

Depots may also be selectively or directionally disposed on abluminal faces, luminal faces, both abluminal and luminal faces, and sidewalls of a stent. Selectively disposing particles in this manner may allow for directional release of the particles and drug release to targeted region. As discussed with reference to FIG. 2A, delivery particles can be released from an abluminal depot into the vascular wall tissue after implantation. Delivery particles are released from luminal depots into the blood stream for treatment of distal vasculature after implantation.

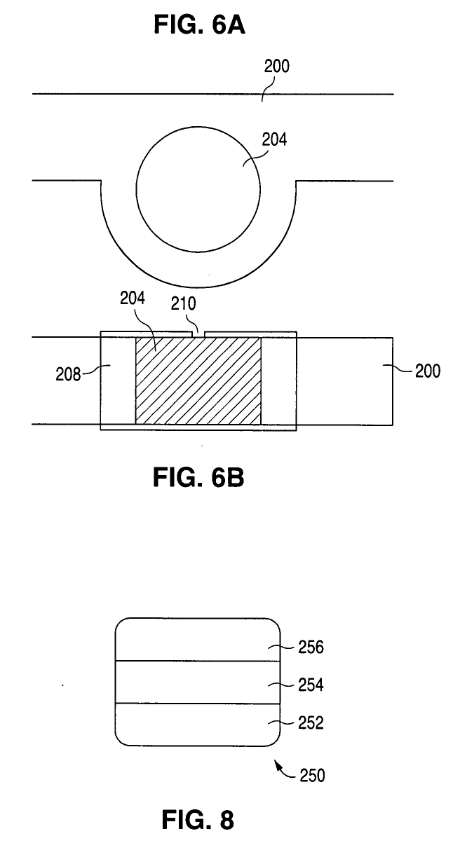

In some embodiments, an active agent for a delivery particle may be released by osmotic pressure. In this embodiment, the active agent or delivery media is disposed in a well cut into a strut of a stent. FIG. 6A depicts an overhead view of a stent strut 200 with a well 204 containing active agent or delivery media. The well may be covered with a coating layer with an opening over well 204. FIG. 6B depicts a side view of strut 200 showing a coating layer 208 disposed above well 204. Coating layer 208 has an opening 210 to allow delivery of active agent or delivery particles from well 204. The difference in concentration of active agent or delivery particles, or an additive such as a salt, in well 204 and outside of well 204 creates an osmotic pressure gradient. This gradient provides for a controlled delivery of active agent or delivery particles through the opening. The opening can be directed either luminally or abluminally.

In further embodiments, an implantable medical device adapted for both local and regional treatment includes a substrate formed from a corrodible metal with a coating including the releasable delivery media that allows for sustained release of active agent(s). The coating can be above at least a portion of the substrate.

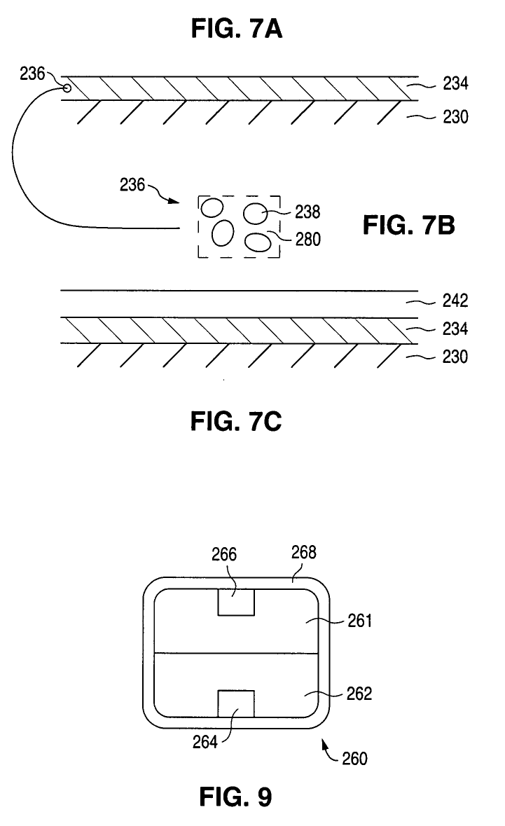

In some embodiments, the coating can include a delivery media, such as particles, dispersed in an erodible binder material. Upon implantation, the erosion or dissolution of the binder causes a release of delivery media, such as particles, into the body. The amount of delivery media can be varied through the ratio of delivery media to binder material. FIG. 7A depicts a delivery media layer 234 over a corrodible metallic substrate 230. FIG. 7B depicts an expanded portion 236 of layer 234 which shows delivery particles 240 dispersed in an erodible binder material 240. As binder material 240 erodes, particles 240 are released into the body and can be transported to distal vasculature for treatment. As depicted in FIG. 7C, an erodible topcoat layer 242 can be disposed above the delivery media coating layer 234 to delay the delivery of the delivery layer. The release of the delivery particles can be controlled by erosion rate of the binder material, the faster the erosion, the faster the release of particles.

In certain embodiments, the coating can be selectively disposed on abluminally or luminally to allow for directional release of delivery media. Referring to FIG. 2, delivery particles can be released from an abluminal layer into the vascular wall tissue after implantation. A luminal coating allows release of drug delivery particles into the blood stream for treatment of distal vasculature after implantation.

In additional embodiments, an implantable medical device adapted for both local and regional treatment includes a substrate formed from an erodible polymer which includes releasable delivery media that allows for sustained release of active agent dispersed within the substrate. As described herein, the delivery media can include particles that are adapted for sustained release of an active agent. A device substrate having dispersed delivery media can be particularly advantageous since it allows release of the delivery media such as particles during all or most of the degradation time of the substrate.

A device substrate with dispersed delivery media can be formed from a polymer construct that is fabricated with dispersed particles. Delivery particles can be blended with a polymer melt and then the melt can be extruded to form a construct, such as a tube. A device can then be formed from the construct, for example, a stent pattern can then be cut into a tube by laser machining the tubing.

In some embodiments, a substrate loaded with delivery particles can also include depots filled with delivery particles or a coating that includes delivery particles. In an embodiment, the substrate can have particles with a different type of agent or drug, or mixture thereof, than a coating or depot. A coating having a different agent or drug, or mixture thereof, can allow staged release of different agents or drugs during different time periods. A depot having a different agent or drug can allow release of different agents or drugs during overlapping time frames.

Any biocompatible polymer suitable for a given treatment may be selected for use in a device, such as a stent. The release profile of delivery media from the substrate can be controlled by the concentration of delivery particles in the substrate and the erosion rate of the erodible polymer. In certain embodiments, the erosion rate of the polymer can be tailored through employment of suitable copolymers and polymer blends. Representative polymers include, but are not limited to, poly(L-lactide), poly(glycolide), poly(DL-lactide), poly(ε-caprolactone), poly(trimethylene carbonate), poly(dioxanone), and copolymers and blends thereof. Exemplary copolymers include, but are not limited to, 90:10 poly(L-Lactide-co-glycolide); 50:50 poly(L-Lactide-co-glycolide); 70:30 poly(L-lactide-co-ε-caprolactone); 70:30 poly(L-lactide-co-DL-lactide); 70:30 poly(L-lactide-co-trimethylene carbonate); and 70:30 poly(L-lactide-co-dioxanone).

In further embodiments, a substrate of a device can have two or more different polymer layers, with at least one layer including dispersed delivery media. In one embodiment, the type of polymers can be the same or different with the layers differing by the type of delivery media. A stent formed with a layered structure can be advantageous, since a layered structure tends to enhance the mechanical stability of a construct.

FIG. 8 depicts a cross-section of strut 250 of a stent with polymer layers 252, 254, and 256. As an example, layers 252 and 256 can have the same type of delivery media while layer 254 has a different delivery media or no delivery media. The polymer of layer 254 may be selected to be stiff and strong to provide mechanical support, while layers 252 and 256 may be selected for to provide flexibility or to provide a selected erosion rate for delivery of the delivery media. Polymer layers can be formed by coextrusion of a tube, followed by cutting of a pattern in the layered tube.

In additional embodiments, the erosion rate of a stent substrate can be modified by including filler materials in the polymer so that it has basic degradation products. When a hydrolytically degradable polymer degrades through hydrolysis, the resulting acidic end groups in the polymer have a tendency to increase the degradation rate through an autocatalytic effect. The influence of basic filler materials on the degradation of amorphous D- and L-lactide copolymer has been previously demonstrated. S. A. T. van der Meer et al., Journal of Materials Science: Materials in Medicine, Volume 7, No. 6, June, 1996. In particular, it was shown that the use of hydroxyapatite as a filler material decreases the degradation rate of the filled polymer. The ability to tune the degradation rate of a polymer system to the clinical need of the system dramatically extends the range of polymers that can be employed in a particular application.

In additional embodiments, a device such as a stent adapted for both local and regional treatment may include a scaffolding with two or more layers, such that at least one layer is a corrodible metal and at least one layer is an erodible polymer. As stated above, a stent formed with a layered structure can be advantageous, since a layered structure tends to enhance the mechanical stability of a construct. A variety of combinations of metal and polymer layers in terms of the number of layers, arrangement of layers, types of material can be envisioned depending on course of treatment desired. A layered scaffolding can have three or more layers with alternating metallic and polymeric layers. The outermost, abluminal and luminal layers, can both be metal, one metal and one polymer, or both polymer.

A scaffolding with metal and polymer layers with drug delivery media can be formed from a tubing with metal and polymer layers. Such tubing can be formed through coextrusion of polymer layers around or within a metal tube, wicking between metallic tubing pieces that are coaxially oriented (one inside other with a clearance in between). Delivery particles can be blended with the polymer melt that is used to form the layers. Additionally, metallic tubes can be dip coated or sprayed to form a coating over the metallic tube. The coating material includes a polymer dissolved in a solvent. Delivery particles can also be included within the coating material. The polymer-coated metallic tube is formed by removing the solvent. The coated metallic tubes can then be slid into each other with the metal surface coated with a solvent or other adhesive agent on the side contacting the polymer. The adhesive can be an adhesive that is activated through heat or vibration. The polymer and metal layers can be uniform in thickness or vary in thickness along the length of the tube. The stent pattern can then be cut by laser machining the tubing.

Embodiments of layered scaffolding can allow for staged release of the delivery media due to differences in degradation rate of the layers. Staged release refers to release of the delivery media over two or more discrete time intervals which may or may not be overlapping. The type of agent and/or drug released in the different time periods can be the same or different.

In some embodiments, a metal or polymer layer can include releasable delivery media, as described above. In one such embodiment, a layer can have depots filled with releasable delivery media. In another such embodiment, the layered structural element can have a coating including releasable delivery media. FIG. 9 depicts a cross-section of a layered strut 260 having a metal abluminal or luminal layer 261 and a polymer abluminal or luminal layer 262. The layers can also include depots 266 and 264 that can be filled with releasable delivery media. A coating layer 268 is disposed above the layers and can act as a top-coat layer or can also include releasable delivery media. In an embodiment, a metal and polymer can be selected so that the polymer erodes faster than the metal layer. Therefore, the metal can provide structural support to the scaffolding during a substantial portion of the time of release of the delivery media.

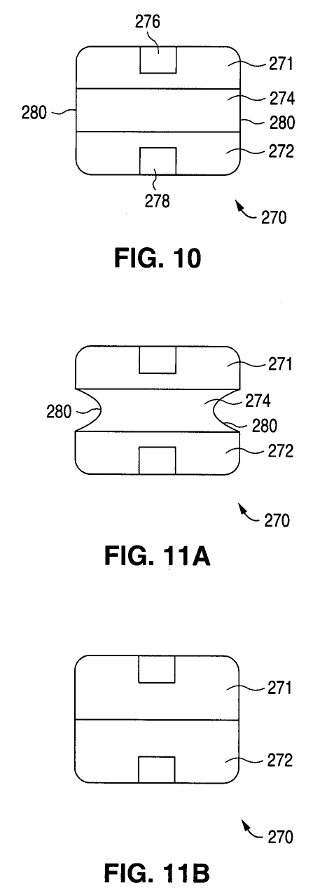

FIG. 10 depicts a three layer strut 270 with metal outer layers 271 and 272 and an inner polymer layer 274. Metal layers 271 and 272 have depots 276 and 278, respectively, filled with releasable delivery media. Polymer layer 274 can have releasable delivery media dispersed within the layer. The release of a the delivery media from the metal layers and the polymer layers may occur in a staged fashion since a majority of the polymer layer 274 is covered by metal layers 270 and 272. Two or more stages of release of the delivery media can be provided by additional inner layers.

It may be desirable to delay the erosion of one or more layers during release of the delivery particles. Delaying the erosion of a layer maintains the mechanical properties of the stent for a longer period of time. Certain embodiments that allow delayed erosion of a layer can include a structural element having an erodible polymer layer between two metallic layers that are not formed of self-dissolving metals. The two metallic layers can be a galvanic couple, such that the metallic layers can undergo galvanic dissolution in bodily fluids when the layers come into contact.

FIG. 10 can be used to illustrate these embodiments. Metallic layers 271 and 272 can be a galvanic couple, which undergo galvanic dissolution in a bodily fluid when in contact. Polymer layer 274 erodes preferentially at sidewalls 280 due to exposure to bodily fluids, as illustrated in FIG. 11A. Additionally, the interior of polymer layer 274 can also erode and the mechanical properties degrade due to diffusion of fluid within polymer layer 274. The degree of diffusion depends on the polymer. Polymers having a high diffusion rate of moisture can be characterized as bulk eroding. Such polymers can exhibit little loss of mass even with a substantial decrease in mechanical properties. The loss of mass and mechanical properties of polymer layer 274 can cause a collapse of polymer layer 274, resulting in contact in between metal layers 270 and 272, as depicted by FIG. 11B. Upon contact, metal layers 270 and 272 undergo galvanic corrosion.

As discussed above, a polymer scaffolding of a stent with dispersed delivery media can be fabricated from tubing formed by melt extrusion with dispersed delivery particles. Additionally, polymer layers of a scaffolding of a stent with dispersed delivery media can be formed from tubing made through coextrusion of the polymer layers.

However, active agents included with drug delivery media may be susceptible to degradation at elevated temperatures. For example, some active agents tend to degrade at temperatures above about 80° C. to 100° C. Thus, it would be desirable to process the polymer and delivery particles at lower temperatures to reduce or prevent degradation of the active agents.

Some embodiments of the present invention can include gel processing of polymers with dispersed delivery media in forming implantable medical devices, such as stents. An important advantage of gel processing is that it allows processing of polymers at temperatures substantially below the melting temperatures of polymers. A “polymer gel” generally refers to a polymer network swollen or capable of being swollen in a liquid. The polymer network can be a network formed by covalent bonds or by physical aggregation with regions of local order acting as network junctions. For example, a physical crosslinked network can be a network of microcrystalline domains in a polymer that act as physical crosslinks or net points.

In some embodiments, the gel can be processed at or near ambient or room temperature. Embodiments can include employing gel processing in fabricating constructs, such as tubes, for stent scaffoldings. Gel processing can also be used to process coatings. In gel processing, a mixture of polymer and solvent that forms a gel is processed.

A representative example of a physically aggregated polymer gel is poly vinyl alcohol (PVA) and swollen with water. In one embodiment, a PVA-water gel is produced from PVA with a high degree of hydrolysis and water. The degree of hydrolysis can be greater than 70%, 80%, or greater than 90%. A gel can be formed by dissolving the PVA in water at a temperature of about 90° C. and then cooling the solution. Gel formation is a function to time, which can be accelerated using a freeze-thaw process. The PVA-water gel includes microcrystalline domains that act as physical cross-links.

Another example of a physically aggregated gel is a block copolymer of poly(L-lactide-glycolic acid) (PLGA) swollen with benzyl benzoate, ethyl benzoate, or benzyl alcohol. Such gels typically are about 50% PLGA and 50% solvent (biocompatible). Such gels can be further include active agents in the range of 10-30%. In some embodiments, a polymer and solvent combination are selected that are capable of forming a gel. The polymer and solvent can be mixed to form a gel in a mixing apparatus, such as a batch mixer or extruder. Active agents, including drug delivery media described above, can be mixed with the gel. The gel mixture can be processed in a forming apparatus such as an extruder to form a polymer construct such as a tube.

The temperature of the gel in the mixing or forming apparatus can be low enough that there is little or no degradation of active agents within the gel. In one embodiment, the temperature is less than a melting temperature of the polymer in the gel, for example, at or about room temperature.

Representative examples of forming apparatuses can include, but are not limited to, single screw extruders, intermeshing co-rotating and counter-rotating twin-screw extruders, and other multiple screw masticating extruders. As the gel is conveyed through the forming apparatus, at least some of the solvent may be vaporized and removed. The gel can then be conveyed through a die to form a polymeric construct, such as a tube.

In certain embodiments, after formation of a construct from the gel, the construct can be dried by removal of some or all of the solvent from the gel. After drying, the construct exhibits the physical properties of the polymer or polymer formulation, but without the solvent that was selected for gelation. In some embodiments, at least some of the solvent in the construct is allowed to remain in the construct. The solvent can elute or diffuse out of the device formed from the construct in vivo upon implantation. In some embodiments, the device is formed from a polymer that does not swell when exposed to bodily fluids. Alternatively, a device can be formed from a polymer that swells upon exposure to bodily fluids.

The formed polymeric part can be dried or cooled by contacting the formed polymeric construct with a cooling fluid having a selected temperature. For example, the formed polymeric construct can be cooled in a quench bath to remove solvent from the gel. Alternatively, the formed polymeric construct may be cooled by air or some other gas at a selected temperature. Some examples of cooling fluids include, but are not limited to, isopropyl alcohol, chloroform, acetone, water, and any mixtures thereof in any proportion.

Representative examples of polymers that may be used for a substrate, binder, coatings, and drug delivery media to fabricate embodiments of implantable medical devices disclosed herein include, but are not limited to, poly(N-acetylglucosamine) (Chitin), Chitosan, poly(3-hydroxyvalerate), poly(lactide-co-glycolide), poly(3-hydroxybutyrate), poly(4-hydroxybutyrate), poly(3-hydroxybutyrate-co-3-hydroxyvalerate), polyorthoester, polyanhydride, poly(glycolic acid), poly(glycolide), poly(L-lactic acid), poly(L-lactide), poly(D,L-lactic acid), poly(D,L-lactide), poly(L-lactide-co-D,L-lactide), poly(caprolactone), poly(L-lactide-co-caprolactone), poly(D,L-lactide-co-caprolactone), poly(glycolide-co-caprolactone), poly(trimethylene carbonate), polyester amide, poly(glycolic acid-co-trimethylene carbonate), co-poly(ether-esters) (e.g. PEO/PLA), polyphosphazenes, biomolecules (such as fibrin, fibrin glue, fibrinogen, cellulose, starch, collagen and hyaluronic acid, elastin and hyaluronic acid), polyurethanes, silicones, polyesters, polyolefins, polyisobutylene and ethylene-alphaolefin copolymers, acrylic polymers and copolymers other than polyacrylates, vinyl halide polymers and copolymers (such as polyvinyl chloride), polyvinyl ethers (such as polyvinyl methyl ether), polyvinylidene halides (such as polyvinylidene chloride), polyacrylonitrile, polyvinyl ketones, polyvinyl aromatics (such as polystyrene), polyvinyl esters (such as polyvinyl acetate), acrylonitrile-styrene copolymers, ABS resins, polyamides (such as Nylon 66 and polycaprolactam), polycarbonates including tyrosine-based polycarbonates, polyoxymethylenes, polyimides, polyethers, polyurethanes, rayon, rayon-triacetate, cellulose, cellulose acetate, cellulose butyrate, cellulose acetate butyrate, cellophane, cellulose nitrate, cellulose propionate, cellulose ethers, and carboxymethyl cellulose. Additional representative examples of polymers that may be especially well suited for use in fabricating embodiments of implantable medical devices disclosed herein include ethylene vinyl alcohol copolymer (commonly known by the generic name EVOH or by the trade name EVAL), poly(butyl methacrylate), poly(vinylidene fluoride-co-hexafluoropropene) (e.g., SOLEF 21508, available from Solvay Solexis PVDF, Thorofare, N.J.), polyvinylidene fluoride (otherwise known as KYNAR, available from ATOFINA Chemicals, Philadelphia, Pa.), ethylene-vinyl acetate copolymers, poly(vinyl acetate), styrene-isobutylene-styrene triblock copolymers, and polyethylene glycol.

Representative examples of biosoluble materials that may be used for a substrate, binder, coatings, and drug delivery media to fabricate embodiments of implantable medical devices disclosed herein include, but are not limited to, poly(ethylene oxide); poly (acrylamide); poly(vinyl alcohol); cellulose acetate; blends of biosoluble polymer with bioabsorbable and/or biostable polymers; N-(2-hydroxypropyl) methacrylamide; and ceramic matrix composites.

Delivery media may incorporate active agent(s) such as anti-inflammatories, antiproliferatives, and other bioactive agents.

An antiproliferative agent can be a natural proteineous agent such as a cytotoxin or a synthetic molecule. Preferably, the active agents include antiproliferative substances such as actinomycin D, or derivatives and analogs thereof (manufactured by Sigma-Aldrich 1001 West Saint Paul Avenue, Milwaukee, Wis. 53233; or COSMEGEN available from Merck) (synonyms of actinomycin D include dactinomycin, actinomycin IV, actinomycin I₁, actinomycin X₁, and actinomycin C₁), all taxoids such as taxols, docetaxel, and paclitaxel, paclitaxel derivatives, all olimus drugs such as macrolide antibiotics, rapamycin, everolimus, structural derivatives and functional analogues of rapamycin, structural derivatives and functional analogues of everolimus, FKBP-12 mediated mTOR inhibitors, biolimus, perfenidone, prodrugs thereof, co-drugs thereof, and combinations thereof. Representative rapamycin derivatives include 40-O-(3-hydroxy)propyl-rapamycin, 40-O-[2-(2-hydroxy)ethoxy]ethyl-rapamycin, or 40-O-tetrazole-rapamycin, 40-epi-(N1-tetrazolyl)-rapamycin (ABT-578 manufactured by Abbot Laboratories, Abbot Park, Ill.), prodrugs thereof, co-drugs thereof, and combinations thereof. In one embodiment, the anti-proliferative agent is everolimus.

An anti-inflammatory drug can be a steroidal anti-inflammatory agent, a nonsteroidal anti-inflammatory agent, or a combination thereof. In some embodiments, anti-inflammatory drugs include, but are not limited to, aldlofenac, aldlometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, deflazacort, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufeniamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, momiflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, aspirin (acetylsalicylic acid), salicylic acid, corticosteroids, glucocorticoids, tacrolimus, pimecorlimus, prodrugs thereof, co-drugs thereof, and combinations thereof. In one embodiment, the anti-inflammatory agent is clobetasol.

Alternatively, the anti-inflammatory may be a biological inhibitor of proinflammatory signaling molecules. Anti-inflammatory biological agents include antibodies to such biological inflammatory signaling molecules.

In addition, the particles and binder may include agents other than antiproliferative agent or anti-inflammatory agents. These active agents can be any agent which is a therapeutic, prophylactic, or a diagnostic agent. In some embodiments, such agents may be used in combination with antiproliferative or anti-inflammatory agents. These agents can also have anti-proliferative and/or anti-inflammatory properties or can have other properties such as antineoplastic, antiplatelet, anti-coagulant, anti-fibrin, antithrombonic, antimitotic, antibiotic, antiallergic, antioxidant, and cystostatic agents. Examples of suitable therapeutic and prophylactic agents include synthetic inorganic and organic compounds, proteins and peptides, polysaccharides and other sugars, lipids, and DNA and RNA nucleic acid sequences having therapeutic, prophylactic or diagnostic activities. Nucleic acid sequences include genes, antisense molecules which bind to complementary DNA to inhibit transcription, and ribozymes. Some other examples of other bioactive agents include antibodies, receptor ligands, enzymes, adhesion peptides, blood clotting factors, inhibitors or clot dissolving agents such as streptokinase and tissue plasminogen activator, antigens for immunization, hormones and growth factors, oligonucleotides such as antisense oligonucleotides and ribozymes and retroviral vectors for use in gene therapy. Examples of antineoplastics and/or antimitotics include methotrexate, azathioprine, vincristine, vinblastine, fluorouracil, doxorubicin hydrochloride (e.g. Adriamycin® from Pharmacia & Upjohn, Peapack N.J.), and mitomycin (e.g. Mutamycin® from Bristol-Myers Squibb Co., Stamford, Conn.). Examples of such antiplatelets, anticoagulants, antifibrin, and antithrombins include sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, thrombin inhibitors such as Angiomax ä (Biogen, Inc., Cambridge, Mass.), calcium channel blockers (such as nifedipine), colchicine, fibroblast growth factor (FGF) antagonists, fish oil (omega 3-fatty acid), histamine antagonists, lovastatin (an inhibitor of HMG-CoA reductase, a cholesterol lowering drug, brand name Mevacor® from Merck & Co., Inc., Whitehouse Station, N.J.), monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), nitric oxide or nitric oxide donors, super oxide dismutases, super oxide dismutase mimetic, 4-amino-2,2,6,6-tetramethylpiperidine-1-oxyl (4-amino-TEMPO), estradiol, anticancer agents, dietary supplements such as various vitamins, and a combination thereof. Examples of such cytostatic substance include angiopeptin, angiotensin converting enzyme inhibitors such as captopril (e.g. Capoten® and Capozide® from Bristol-Myers Squibb Co., Stamford, Conn.), cilazapril or lisinopril (e.g. Prinivil® and Prinzide® from Merck & Co., Inc., Whitehouse Station, N.J.). An example of an antiallergic agent is permirolast potassium. Other therapeutic substances or agents which may be appropriate include alpha-interferon, and genetically engineered epithelial cells. The foregoing substances are listed by way of example and are not meant to be limiting.

Other bioactive agents may include antiinfectives such as antiviral agents; analgesics and analgesic combinations; anorexics; antihelmintics; antiarthritics, antiasthmatic agents; anticonvulsants; antidepressants; antidiuretic agents; antidiarrheals; antihistamines; antimigrain preparations; antinauseants; antiparkinsonism drugs; antipruritics; antipsychotics; antipyretics; antispasmodics; anticholinergics; sympathomimetics; xanthine derivatives; cardiovascular preparations including calcium channel blockers and beta-blockers such as pindolol and antiarrhythmics; antihypertensives; diuretics; vasodilators including general coronary; peripheral and cerebral; central nervous system stimulants; cough and cold preparations, including decongestants; hypnotics; immunosuppressives; muscle relaxants; parasympatholytics; psychostimulants; sedatives; tranquilizers; naturally derived or genetically engineered lipoproteins; and restenoic reducing agents. Other active agents which are currently available or that may be developed in the future are equally applicable.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

1. A stent comprising a scaffolding formed from a corrodible metal having one or more recesses in a surface of the scaffolding, the recesses being at least partially filled with a plurality of releasable delivery media comprising an active agent, wherein the active agent is adapted to be released from the delivery media upon release of the delivery media from an implanted stent.

2. The method of claim 1, wherein the delivery media allows for sustained-release of active agent into a body of a patient upon release of the delivery media from the implanted stent.

3. The method of claim 1 wherein the metal is porous.

4. The method of claim 1 wherein the metal has a porosity of at least 50%.

5. The method of claim 1 wherein the metal dissolves upon exposure to bodily fluids.

6. The method of claim 1 wherein the metal comprises a combination of two or more metals selected to create a galvanic couple such that the metal undergoes galvanic dissolution upon contact with bodily fluids.

7. The method of claim 1 wherein the recesses comprise a plurality of depots in the surface of the substrate.

8. The method of claim 1 wherein the recesses are on a luminal surface of the stent.

9. The method of claim 1 wherein the recesses are on an abluminal surface of the stent.

10. The method of claim 1 wherein the delivery media are mixed or dispersed in an erodible polymer, wherein at least some of the delivery media are released from the implanted stent upon erosion of the erodible polymer.

11. The method of claim 1 further comprising an erodible coating above the opening of the depots, the coating adapted to delay the release of the delivery media from the implanted stent.

12. The method of claim 1 wherein the delivery media comprise nanoparticles, wherein an active agent is encapsulated within, coated on, or dispersed within the nanoparticles.

13. A stent comprising a scaffolding including at least two erodible polymer layers, wherein at least one of the polymer layers comprises a plurality of delivery media comprising an active agent, wherein the active agent is adapted to be released from the delivery media upon erosion of the at least one polymer layer.

14. The stent of claim 13 wherein the delivery media allows for sustained-release of the active agent into a body of a patient upon release of the delivery media from the implanted stent.

15. The stent of claim 13 wherein the plurality of delivery media comprise a plurality of particles comprising the active agent.

16. The stent of claim 13 wherein at least two of the polymer layers comprise the same delivery media.

17. The stent of claim 13 wherein at least two of the polymer layers comprise different delivery media.

18. The stent of claim 13 wherein at least one of the polymer layers comprises a polymer having a greater stiffness than at least one of the other polymer layers.

19. The stent of claim 13 wherein at least one of the polymer layers comprises a filler material that modifies an erosion rate of the scaffolding.

20. The stent of claim 19 wherein the filler has basic degradation products that decrease the erosion rate of the stent scaffolding.

21. The stent of claim 20 wherein the filler is hydroxyapatite.

22. A stent comprising a scaffolding including at least two erodible polymer layers, wherein at least one of the polymer layers comprises a plurality of delivery media comprising an active agent, wherein the active agent is adapted to be released from the delivery media upon erosion of the at least one polymer layer, wherein at least one of the polymer layers comprises a filler material that modifies an erosion rate of the scaffolding, wherein the filler has basic degradation products that decrease the erosion rate of the stent scaffolding.

23. A stent comprising a scaffolding including struts having an abluminal layer, a luminal layer, and a middle layer between the abluminal layer and the luminal layer, each of the layers being formed from erodible polymers, wherein a plurality of delivery media are dispersed within the abluminal layer or luminal layer, wherein an active agent is adapted to be released from the delivery media upon release of the delivery media from the scaffolding of an implanted stent during erosion of the abluminal layer or luminal layer releases the delivery media from the scaffolding.

24. The stent of claim 23 wherein the middle layer comprises a polymer having a greater stiffness than the abluminal and luminal polymer layers, thereby providing structural support to the scaffolding.

25. The stent of claim 23 wherein at least one of the polymer layers comprises a filler material that modifies an erosion rate of the scaffolding.

26. The stent of claim 25 wherein the filler has basic degradation products that decrease the erosion rate of the stent scaffolding.

27. The stent of claim 26 wherein the filler is hydroxyapatite.

28. A stent comprising erodible polymer struts having an abluminal layer, a luminal layer, and a middle layer, wherein a plurality of delivery media comprising an active agent are dispersed within the abluminal layer or luminal layer, wherein the active agent is adapted to be released from the delivery media upon release of the delivery media from the scaffolding, wherein erosion of the abluminal layer or luminal layer releases the delivery media from the scaffolding, wherein at least one of the layers comprises a filler material that modifies an erosion rate of the scaffolding, wherein the filler has basic degradation products that decrease the erosion rate of the stent scaffolding, and wherein the filler is hydroxyapatite.

29. A method of fabricating a stent comprising:
co-extruding a tube including at least two erodible polymer layers, wherein at least one of the two erodible polymer layers comprises a plurality of delivery media comprising an active agent; and
cutting a stent pattern in the tube to form a stent scaffolding including at least two erodible polymer layers, wherein the active agent is adapted to be released from the delivery media upon release of the delivery media from the polymer layer of an implanted stent due to erosion of the at least two polymer layers.

30. The method of claim 29 wherein the plurality of delivery media comprise a plurality of particles comprising the active agent.

31. A stent comprising a scaffolding having an erodible polymer layer and an erodible metal layer, wherein at least one of the layers comprises a plurality of delivery media comprising an active agent, wherein the active agent is adapted to be released from the delivery media upon release of the delivery media from the scaffolding of an implanted stent due to erosion of one of the scaffolding layers.

32. The stent of claim 31 wherein one of the layers is an abluminal layer and the other layer is a luminal layer.

33. The stent of claim 31 wherein the delivery media allows for sustained-release of an active agent into a body of a patient upon release of the delivery media from the scaffolding of an implanted stent.

34. The stent of claim 31 wherein the polymer layer comprises the delivery media.

35. The stent of claim 31 wherein the metal layer comprises the delivery media.

36. The stent of claim 31 wherein the erosion rate of the metal and polymer layers is different, allowing for staged release of an active agent into a body of a patient upon release of the delivery media.

37. The stent of claim 31 wherein the delivery media are disposed within a recess in a surface of at least one of the layers.

38. The stent of claim 31 wherein the delivery media are dispersed within the polymer layer.

39. The stent of claim 31 wherein the delivery media are dispersed within an erodible coating disposed above at least one of the layers.

40. The stent of claim 31 wherein the metal layer is formed from a metal selected from the group consisting of magnesium, manganese, potassium, calcium, sodium, zinc, chromium, iron, cadmium, aluminum, cobalt, vanadium, copper, molybdenum, antimony, and alloys thereof.

41. A stent comprising a scaffolding having an erodible polymer layer and an erodible metal layer, wherein at least one of the layers comprises a plurality of delivery media comprising an active agent, wherein the active agent is adapted to be released from the delivery media upon release of the delivery media from the scaffolding of an implanted stent due to erosion of one of the layers, and wherein the metal layer is formed from a metal selected from the group consisting of magnesium, manganese, potassium, calcium, sodium, zinc, chromium, iron, cadmium, aluminum, cobalt, vanadium, copper, molybdenum, antimony, and alloys thereof.

42. A stent comprising a scaffolding including an erodible polymer layer between two erodible metal layers, at least one of the layers comprising a plurality of releasable delivery media comprising an active agent, wherein the active agent is adapted to be released from the delivery media upon release of the delivery media from an implanted stent.

43. The stent of claim 42 wherein the metallic layers comprise an abluminal layer and a luminal layer.

44. The stent of claim 42 wherein the metal layers delay release of the delivery media from the polymer layer allowing for staged release of the active agents into a body of a patient upon release of the delivery media from the metal and polymer layers.

45. The stent of claim 42 wherein the metal layers provide structural support during release of the delivery media from the polymer layer.

46. The stent of claim 42 wherein erosion of the metal layers is delayed by the polymer layer.

47. The stent of claim 42 wherein the metal layers are self-dissolving.

48. The stent of claim 30 wherein the metal layers are a galvanic couple that undergo galvanic erosion upon contact.

49. The stent of claim 42 wherein the delivery media allow for sustained-release of active agent into a body of a patient upon release of the delivery media from the implanted stent.

50. The stent of claim 42 wherein the metal layers are formed from a metal selected from the group consisting of magnesium, manganese, potassium, calcium, sodium, zinc, chromium, iron, cadmium, aluminum, cobalt, vanadium, copper, molybdenum, antimony, and alloys thereof.

51. A method of fabricating a stent comprising:
forming a layered tube comprising an erodible polymer layer within or around an erodible metallic tube, wherein the erodible polymer layer comprises a plurality of delivery media comprising an active agent; and
cutting a stent pattern in the layered tube to form a stent scaffolding, the stent scaffolding having an erodible metallic layer and an erodible polymer layer, wherein the active agent is adapted to be released from the delivery media upon release of the delivery media from the scaffolding of an implanted stent.

52. The method of claim 51 wherein the erodible polymer layer is formed by co-extruding the erodible polymer layer within or around the erodible metallic tube.

53. The method of claim 51 wherein the erodible polymer layer is formed by coating the erodible metallic tube.

54. The method of claim 51 further comprising forming a cavity in the erodible metallic layer of the scaffolding and disposing a plurality of delivery media within the cavity.

55. A method of fabricating a stent comprising:
forming a layered tube comprising an erodible polymer layer within or around an erodible metallic tube, wherein the erodible polymer layer comprises a plurality of delivery media comprising an active agent;
cutting a stent pattern in the layered tube to form a stent scaffolding, the stent scaffolding having an erodible metallic layer and an erodible polymer layer, wherein the active agent is adapted to be released from the delivery media upon release of the delivery media from the scaffolding of an implanted stent;
forming a cavity in the erodible metallic layer of the scaffolding; and
disposing a plurality of delivery media within the cavity.

56. A method of forming a stent, comprising:
forming a gel mixture comprising an erodible polymer, solvent, and a plurality of delivery media, the delivery media comprising an active agent;
processing the gel mixture to form a tube, the erodible polymer and the delivery media dispersed within the tube; and
forming a stent from the tube.

57. The method of claim 56 wherein the gel mixture is processed at or near room temperature.

58. The method of claim 56 wherein the erodible polymer comprises poly vinyl alcohol (PVA) or a block copolymer of poly(L-lactide-glycolic acid)(PLGA).

59. The method of claim 56 wherein the solvent is selected from the group consisting of water, benzyl benzoate, ethyl benzoate, and benzyl alcohol.

60. The method of claim 56 wherein the gel mixture is formed in a mixing apparatus selected from the group consisting of a batch mixer and an extruder.

61. The method of claim 56 wherein the active agent is adapted to be released from the delivery media upon release of the delivery media from the implanted stent.

62. The method of claim 56 wherein the processing comprises extruding the gel mixture to form the tube.

63. The method of claim 56 wherein the solvent is removed from the gel mixture during and after forming the tube.

64. The method of claim 63 wherein the solvent is removed by cooling the gel mixture with a cooling fluid.

65. A method of forming a stent, comprising:
forming a gel mixture comprising an erodible polymer, solvent, and a plurality of delivery media, the delivery media comprising an active agent;
processing the gel mixture to form a tube, the erodible polymer and the delivery media dispersed within the tube;
forming a stent from the tube; and
removing the solvent from the gel mixture during and after forming the tube by cooling the gel mixture with a cooling fluid.

66. The method of claim 65 wherein the processing comprises coextruding the gel mixture around or within a polymer or metallic tube to form the tube, wherein the tube comprises a layer formed from the gel mixture and a layer comprising the polymer or metallic tube.

67. A method of forming a stent, comprising:
forming a gel mixture comprising an erodible polymer, solvent, and a plurality of delivery media, the delivery media comprising an active agent;
processing the gel mixture to form a tube, the erodible polymer and the delivery media dispersed within the tube, wherein the processing comprises coextruding the gel mixture around or within a polymer or metallic tube to form the tube, wherein the tube comprises a layer formed from the gel mixture and a layer comprising the polymer or metallic tube; and
forming a stent from the tube.

68. The method of claim 67 wherein the stent is formed by cutting a stent pattern in the tube.

69. A method of forming a stent, comprising:
forming a gel mixture comprising an erodible polymer, solvent, and a plurality of delivery media comprising an active agent;
fabricating a tube from the gel mixture with a forming apparatus, the tube including a layer comprising the erodible polymer and the delivery media dispersed within the layer; and
forming a stent from the tube.

70. The method of claim 69 wherein the gel mixture is processed at or near room temperature.

71. The method of claim 69 wherein the forming apparatus comprises an extruder.

72. The method of claim 69 wherein the forming apparatus comprises an extruder and a die.

73. The method of claim 69 wherein the gel mixture is coextruded around or within a polymer or metallic tube to form the tube, wherein the tube comprises the layer formed from the gel mixture and a layer comprising the polymer or metallic tube.

74. The method of claim 69 wherein the active agent is adapted to be released from the delivery media upon release of the delivery media from the stent upon implantation.

75. A method of forming a stent, comprising:
forming a gel mixture comprising an erodible polymer, solvent, and a plurality of delivery media comprising an active agent, wherein the active agent is adapted to be released from the delivery media upon release of the delivery media from the stent upon implantation;
fabricating a tube from the gel mixture with a forming apparatus, the tube including a layer comprising the erodible polymer and the delivery media dispersed within the layer; and
forming a stent from the tube.

76. A stent comprising a structural element having a cavity disposed therein including a plurality of releasable delivery media comprising an active agent, wherein an osmotic pressure gradient between the cavity and the surface of the structural element releases the delivery media from the cavity.

77. The stent of claim 76 wherein the active agent is adapted to be released from the delivery media upon release of the delivery media from the implanted stent.

78. The stent of claim 76 wherein the opening is on an abluminal surface or a luminal surface of the structural element.

79. The stent of claim 76 wherein the structural element comprises a coating layer covering the cavity, an opening being through the coating layer.

80. The stent of claim 79 wherein the coating is erodible.

81. A stent comprising a structural element having a cavity disposed therein including a plurality of releasable delivery media, wherein an osmotic pressure gradient between the cavity and a surface of the structural element releases the delivery media from the cavity, wherein the structural element comprises a coating layer covering the cavity, an opening being through the coating layer, and wherein the coating is erodible.

82. The stent of claim 76 wherein the osmotic pressure gradient is formed by a difference in concentration of the delivery media or an active agent in the cavity and at the surface of the structural element.

83. The stent of claim 76 wherein the osmotic pressure gradient is formed by a difference in concentration of an additive in the cavity and at the surface of the structural element.

84. The stent of claim 83 wherein the additive is a salt.

85. A stent comprising a structural element having a cavity disposed therein including a plurality of releasable delivery media, wherein an osmotic pressure gradient between the cavity and a surface of the structural element releases the delivery media from the cavity, wherein the osmotic pressure gradient is formed by a difference in concentatrion of an additive in the cavity and at the surface of the structural element, and wherein the additive is a salt.

86. The stent of claim 76 wherein the structural element is formed from an erodible metal.

87. A stent comprising a structural element having a cavity disposed therein including a plurality of releasable delivery media comprising an active agent, wherein an opening between the cavity and a surface of the structural element, wherein an osmotic pressure gradient between the cavity and the surface of the structural element releases the delivery media through the opening from an implanted stent, and wherein the structural element is formed from an erodible metal.

88. The stent of claim 76 wherein the structural element is formed from a metal selected from the group consisting of magnesium, manganese, zinc, chromium, iron, aluminum, cobalt, tin, vanadium, copper, and molybdenum.

89. A stent comprising a structural element having a cavity disposed therein including a plurality of releasable delivery media comprising an active agent, wherein an osmotic pressure gradient between the cavity and a surface of the structural element releases the delivery media through an opening between the cavity and the surface of the structural element, and wherein the structural element is formed from a metal selected from the group consisting of magnesium, manganese, zinc, chromium, iron, aluminum, cobalt, tin, vanadium, copper, and molybdenum.

90. A stent comprising an erodible scaffolding, the scaffolding comprising a plurality of releasable particles, wherein the particles comprise an active agent and are adapted to be released from the stent upon erosion of the scaffolding.

91. The stent of claim 90 wherein the active agent is adapted to be released from the particles upon release of the particles from the scaffolding.

92. The stent of claim 90 wherein the particles are nanoparticles.

93. The stent of claim 90 wherein the particles are incorporated on or within the scaffolding with an erodible binder, the binder holding the particles together on or within the scaffolding.

94. The stent of claim 90 wherein the particles are disposed within a recess in a surface of the scaffolding.

95. The stent of claim 90 wherein the particles are dispersed within an erodible binder disposed above the surface of the scaffolding.

96. The stent of claim 90 wherein the active agent is encapsulated within, coated on, or dispersed within the particles.

97. The stent of claim 90 wherein at least a portion of the scaffolding is formed from an erodible polymer.

98. The stent of claim 90 wherein at least a portion of the scaffolding is formed from an erodible metal.

99. The stent of claim 90 wherein the particles are formed from a precipitate of a neat bioactive agent.

100. The stent of claim 90 wherein the particles comprise a polymer and a drug.

101. The stent of claim 90 wherein the particles comprise a drug impregnated core and a bioerodible coating.

102. The stent of claim 90 wherein the particles comprise a fullerene with a bioactive agent coating.

103. The stent of claim 90 wherein the particles are selected from the group consisting of polymerosome, micelle, vesicle, liposome, biodegradable glass, biostable glass, carbon nanotube and micronized drug.

104. The stent of claim 90 wherein the particles are formed from a material selected from the group consisting of bioabsorbable polymer, biostable polymer, biosoluble material, biopolymer, biostable metal, biocrodible metal, block copolymer of a bioabsorbable polymer, block copolymer of a biopolymer, ceramic, salt, lipid, and a combination thereof.

105. The stent of claim 90 wherein a surface of the particles are adapted to bind to a portion of vasculature.

106. The stent of claim 90 wherein a surface of the particles comprises a substance incorporated into the surface for selectively binding the surface to a portion of the vasculature, the substance selected from the group consisting of a peptide, an antibody, a small-molecular ligand, and a specific receptor having an affinity to receptors found on endothelial cells.

107. A stent comprising an erodible scaffolding, the scaffolding comprising a plurality of releasable particles, wherein the particles comprise an active agent and are adapted to be released from the stent upon erosion of the scaffolding, and wherein the active agent is adapted to be released from the particles upon release of the particles from the scaffolding.

108. A stent comprising an erodible scaffolding, the scaffolding comprising a plurality of releasable particles, wherein the particles comprise an active agent and are adapted to be released from the stent upon erosion of the scaffolding, and wherein the particles are incorporated on or within the scaffolding with an erodible binder, the binder holding the particles together on or within the scaffolding.

109. A stent comprising an erodible scaffolding, the scaffolding comprising a plurality of releasable particles, wherein the particles comprise an active agent and are adapted to be released from the stent upon erosion of the scaffolding, and wherein the particles are dispersed within an erodible binder disposed above the surface of the scaffolding.

110. A stent comprising an erodible scaffolding, the scaffolding comprising a plurality of releasable particles, wherein the particles comprise an active agent and are adapted to be released from the stent upon erosion of the scaffolding, and wherein the active agent is encapsulated within, coated on, or dispersed within the particles.

111. A method of treating a patient vasculature, comprising:
deploying a stent at an implant site of a vasculature, the stent comprising a scaffolding formed from erodible material, the scaffolding comprising a plurality of releasable delivery media comprising an active agent; and
allowing the delivery media to release from the scaffolding and be transported to a target region of the vasculature.

112. The method of claim 111 further comprising allowing the delivery media to bind to the target region of the vasculature.

113. The method of claim 111 wherein a surface of the delivery media is adapted to bind to the target region of the vasculature.

114. The method of claim 111 wherein a surface of the delivery media comprises a substance incorporated into the surface for selectively binding the surface to a portion of the vasculature, the substance selected from the group consisting of a peptide, an antibody, a small-molecular ligand, and a specific receptor having an affinity to receptors found on endothelial cells.

115. The method of claim 111 wherein the erodible material comprises an erodible polymer, erodible metal, or a combination thereof.

116. The method of claim 111 wherein the active agent is released from the delivery media upon release of the delivery media from the scaffolding.

117. The method of claim 111 wherein the active agent is released from the delivery media during transport and at the target region of the vasculature.

118. The method of claim 111 wherein the released delivery media provide sustained-release of the active agent into the vasculature.

119. The method of claim 111 wherein the delivery media is released from the scaffolding due to erosion of the erodible material.

120. The method of claim 111 wherein the delivery media is released into the vasculature and transported within the blood.

121. The method of claim 111 wherein the delivery media is released into and transported through the vascular wall to the target region.

122. The method of claim 111 wherein the delivery media is released from a luminal surface of the scaffolding.

123. The method of claim 111 wherein the delivery media is released from an abluminal surface of the scaffolding.

124. The method of claim 111 wherein the delivery media is released from a surface between an abluminal surface and a luminal surface of the scaffolding.

125. The method of claim 111 wherein the target region of the vasculature is distal to the implant site of the stent.

126. The method of claim 111 wherein the target region of the vasculature is proximal to the scaffolding.

127. The method of claim 111 wherein the delivery media are incorporated on or within the scaffolding with an erodible binder, the binder holding the particles together on or within the scaffolding.

128. The method of claim 127 wherein the binder comprises a biodegradable polymer or a water soluble polymer.

129. A method of treating a patient vasculature, comprising:
deploying a stent at an implant site of a vasculature, the stent comprising a scaffolding formed from erodible material, the scaffolding comprising a plurality of releasable delivery media comprising an active agent, wherein the delivery media are incorporated on or within the scaffolding with an erodible binder, the binder holding the particles together on or within the scaffolding, and wherein the binder comprises a biodegradable polymer or a water soluble polymer; and
allowing the delivery media to release from the scaffolding and be transported to a target region of the vasculature.

130. The method of claim 129 wherein the delivery media are disposed within a recess in a surface of the scaffolding.

131. The method of claim 129 wherein the delivery media are dispersed within the erodible binder disposed above the surface of the scaffolding.

132. A method of treating a patient vasculature, comprising:
deploying a stent at an implant site of a vasculature, the stent comprising a scaffolding formed from an erodible material, the scaffolding comprising a plurality of releasable delivery media comprising an active agent, and wherein a surface of the delivery media is adapted to bind to the target region of the vasculature; and
allowing the delivery media to release from the scaffolding and be transported to a target region of the vasculature.

133. A method of treating a patient vasculature, comprising:
deploying a stent at an implant site of a vasculature, the stent comprising a scaffolding formed from erodible material, the scaffolding comprising a plurality of releasable delivery media comprising an active agent, and wherein a surface of the delivery media comprises a substance incorporated into the surface for selectively binding the surface to a portion of the vasculature, the substance selected from the group consisting of a peptide, an antibody, a small-molecular ligand, and a specific receptor having an affinity to receptors found on endothelial cells; and
allowing the delivery media to release from the scaffolding and be transported to a target region of the vasculature.

134. A method of treating a patient vasculature, comprising:
deploying a stent at an implant site of a vasculature, the stent comprising a scaffolding formed from erodible material, the scaffolding comprising a plurality of releasable delivery media comprising an active agent, and wherein the delivery media are incorporated on or within the scaffolding with an erodible binder, the binder holding the particles together on or within the scaffolding; and
allowing the delivery media to release from the scaffolding and be transported to a target region of the vasculature.

135. A stent comprising:
a scaffolding formed from an erodible material; and
a delivery coating disposed over at least a portion of the scaffolding, the delivery coating comprising a releasable delivery media dispersed within an erodible binder material, wherein the binder material is adapted to erode and release the delivery media upon implantation of the stent.

136. The stent of claim 135 wherein the delivery coating is selectively disposed over an abluminal surface or a luminal surface of the scaffolding.

137. The stent of claim 135 wherein the erodible material is an erodible polymer.

138. The stent of claim 135 wherein the erodible material is a corrodible metal.

139. The stent of claim 135 wherein the binder material is selected from the group consisting of a bioabsorbable polymer and a biosoluble polymer.

140. The stent of claim 135 wherein the delivery media comprises an active agent, the delivery media allowing for sustained-release of the active agent into a body of a patient upon release of the delivery media from the binder material.

141. The stent of claim 135 further comprising an erodible top coating above the delivery coating, the top coating adapted to delay the release of the delivery media from the delivery coating.

142. A stent comprising:
a scaffolding formed from an erodible material;
a delivery coating disposed over at least a portion of the scaffolding, the delivery coating comprising a releasable delivery media dispersed within an erodible binder material, wherein the binder material is adapted to erode and release the delivery media upon implantation of the stent, and wherein the delivery coating is selectively disposed over an abluminal surface or a luminal surface of the scaffolding.

143. A stent comprising:
a scaffolding formed from an erodible material;
a delivery coating disposed over at least a portion of the scaffolding, the delivery coating comprising a releasable delivery media dispersed within an erodible binder material, wherein the binder material is adapted to erode and release the delivery media upon implantation of the stent, and wherein the delivery media comprises an active agent, the delivery media allowing for sustained-release of the active agent into a body of a patient upon release of the delivery media from the binder material.

144. A method of fabricating a coated stent comprising:
applying a coating material to a stent scaffolding, the coating material comprising an erodible polymer dissolved in a solvent and a plurality of delivery media dispersed in the solvent; and
removing all or substantially all of the solvent to form a delivery coating over the scaffolding, the delivery coating comprising the plurality of delivery media dispersed in the erodible polymer, wherein the erodible polymer is adapted to erode and release the delivery media upon implantation of the stent.

145. The method of claim 144 wherein the delivery coating is applied by spraying the coating material on the scaffolding or dipping the scaffolding in the coating material.

146. The method of claim 144 wherein the coating material is selectively disposed over an abluminal surface or a luminal surface of the scaffolding to form an abluminal or luminal coating.

147. The method of claim 144 wherein the stent scaffolding is formed from an erodible material.

148. The method of claim 147 wherein the erodible material is an erodible polymer.

149. The method of claim 147 wherein the erodible material is a corrodible metal.

150. The method of claim 144 wherein the coating polymer is selected from the group consisting of a bioabsorbable polymer and a biosoluble polymer.

151. The method of claim 144 wherein the delivery media comprises an active agent, the delivery media allowing for sustained-release of the active agent into a body of a patient upon release of the delivery media from the delivery coating.

152. The method of claim 144 further comprising forming an erodible top coating above the delivery coating, the top coating adapted to delay the release of the delivery media from the delivery coating.

153. A method of fabricating a coated stent comprising:
applying a coating material to a stent scaffolding, the coating material comprising an erodible polymer dissolved in a solvent and a plurality of delivery media dispersed in the solvent;
removing all or substantially all of the solvent to form a delivery coating over the scaffolding, the delivery coating comprising the plurality of delivery media dispersed in the erodible polymer, wherein the erodible polymer is adapted to erode and release the delivery media upon implantation of the stent; and
forming an erodible top coating above the delivery coating, the top coating adapted to delay the release of the delivery media from the delivery coating.
